(19) United States
(12) Patent Application Publication
(10) Pub. No.: US 2024/0316339 A1
(43) Pub. Date: Sep. 26, 2024

(54) WRISTBAND NERVE STIMULATOR AND USE METHOD THEREOF

(71) Applicants: Fasikl Incorporated, Dallas, TX (US); Hangzhou Fasikl Technology Co., Ltd., Hangzhou (CN)

(72) Inventors: Edward Keefer, DALLAS, TX (US); Jules Anh Tuan Nguyen, DALLAS, TX (US); Bing Ye, Hangzhou (CN); Baitong Wang, Hangzhou (CN); Markus Drealan, DALLAS, TX (US)

(21) Appl. No.: 18/125,778

(22) Filed: Mar. 24, 2023

(51) Int. Cl. A61N 1/04; A61N 1/36

(52) U.S. Cl. CPC A61N 1/0456; A61N 1/0476; A61N 1/36067

(57) ABSTRACT

The present disclosure discloses a wristband nerve stimulator and a use method thereof. The wristband nerve stimulator is small in volume and wearable, and has a waterproof function. As a wristband instrument, the wristband nerve stimulator can perform treatment at any time. Furthermore, the fixed main unit and the band can be quickly mounted and removed, which is convenient to replace the band.

### TECHNICAL FIELD

The present disclosure relates to the technical field of medical instruments, specifically to a wristband nerve stimulator and a use method thereof.

### BACKGROUND

Electrical energy can be delivered through the skin via electrodes on a skin surface using a nerve stimulation system to stimulate peripheral nerves, as a non-limiting example, such as the median, radialis, and/or ulnar nerves in the upper limbs. It has been shown that the electrical stimulation for these nerves provides therapeutic benefits for various diseases including but not limited to dyskinesia (including but not limited to essential tremor, Parkinson's tremor, postural tremor and multiple sclerosis), urological diseases, gastrointestinal diseases, heart diseases and inflammatory diseases.

Percutaneous electrical nerve stimulation belongs to an electrical stimulation therapy for peripheral nerves. Because of its advantages of no trauma, non-invasiveness, easy operation, good tolerance, short effect, no side effect, low price and the like, the percutaneous electrical nerve stimulation has become a hot spot in clinical application researches, and is widely used, such as various electrical nerve stimulation therapeutic apparatuses based on the percutaneous electrical nerve stimulation.

A traditional nerve stimulator has a large volume, and cannot be carried or is inconvenient to carry, so that treatment cannot be performed at any time; personalized configuration cannot be achieved according to personal actual conditions; the wearing process is complex and troublesome, and the traditional nerve stimulator is very inconvenient to wear: doctors' specialized guidance is required during use; and the traditional nerve stimulator cannot be used in daily lives of families and is difficult for wide promotion. Electrode pieces that contact the skin are easy to abrade due to long-term friction with the skin. Parameters of an abraded electrode piece change, affecting the treatment effect. Therefore, the electrode pieces are consumables.

### SUMMARY

The present disclosure aims to provide a wristband nerve stimulator and a use method thereof. The wristband nerve stimulator has the advantages of small volume and convenient wearing, and solves the problems in the background section.

In order to achieve the above objective, the present disclosure provides the following technical solution: A wristband nerve stimulator includes:
a fixed main unit, wherein the fixed main unit includes a shell, a circuit assembly and a screen assembly; the circuit assembly is electrically connected with the screen assembly; the screen assembly and the shell are encapsulated through a waterproof sealing rubber strip; the circuit assembly is fixedly mounted in the shell; the circuit assembly includes a lithium battery, a circuit board; and a vibration motor arranged on the circuit board; a plurality of first threaded holes and a framework matched with the vibration motor are arranged in the shell; the vibration motor is embedded into the framework; a plurality of second threaded holes corresponding to the first threaded holes are formed in four corners of the circuit board; the plurality of first threaded holes are fixedly connected with the plurality of second threaded holes through screws; the first threaded holes are all blind holes; a charging interface and an adapter port are arranged on the circuit board; a bottom of the shell is provided with a charging interface location and an adapter port location which are matched with the charging interface and the adapter port; the charging interface and the adapter port are respectively embedded into the charging interface location and the adapter port location: spaces between the charging interface and the charging interface location as well as between the adapter port and the adapter port location are encapsulated by sealants; the bottom of the shell is also provided with a plurality of magnetic attraction slots; a magnet is embedded into each magnetic attraction slot;
a band, wherein the band includes an outer band and an inner band; the inner band includes a base and an electrode assembly; the base is connected with the electrode assembly; the electrode assembly includes a plurality of electrode pieces and an adapter seat; the plurality of electrode pieces are sequentially connected through a flexible printed circuit (FPC) flat cable; the electrode piece at the outermost end is arranged at the bottom of the base and protrudes from the bottom of the base, and sealing layers wrap outer sides of the remaining electrode pieces and the FPC flat cable; the electrode pieces on the sealing layers protrude from inner walls of sealing strips; an open fastener is arranged on one side of each sealing layer away from the base; the fastener is fastened on the outer band; the adapter seat is arranged on the base; the adapter seat is electrically connected with the FPC flat cable; the base is also provided with a plurality of circular magnetic attraction slots matched with the plurality of magnetic attraction slots; a magnet is embedded in each circular magnetic attraction slot; the magnets in the plurality of magnetic attraction slots are connected to the magnets in the plurality of circular magnetic attraction slots in an attracting manner; the adapter seat is embedded into the adapter port; the circuit assembly is electrically connected with the electrode assembly; two ends of the outer band are movably connected with front and rear sides of the fixed main unit; the outer band and the fixed main unit form a ring; and the ring wraps the inner band.

Preferably, the screen assembly includes a display screen and a display interface flat cable; the display screen is connected with the circuit board through the display interface flat cable; a glass cover plate is covered on the display screen; and a transparent frosted region is arranged at one corner of the glass cover plate.

Preferably, a plurality of micro switches are arranged at a side end of the circuit board; a plurality of buttons matched with the micro switches are arranged at a side end of the shell; the buttons and the shell linearly slide and are in clearance fit; a reset spring and a waterproof sealing ring are arranged between the buttons and the shell; the plurality of micro switches resist against the plurality of buttons; a charging abutment contact and an adapter port abutment contact are also arranged on the circuit board; the charging abutment contact is connected with the charging interface; a plurality of pogopins are arranged on the adapter port; the pogopins on the adapter port are connected with the adapter port abutment contact in a contacting manner; and two magnets are embedded in the charging interface.

Preferably, the circuit board includes a microprocessor, a circuit management unit, a communication unit, a tremor detection unit, a button and display unit, a vibration unit, a data storage unit, an inertial sensing unit, an ambient light detection circuit, a wristband detection circuit, an impedance detection circuit and an electrical stimulation circuit; the circuit management unit, the communication unit, the tremor detection unit, the button and display unit, the vibration unit, the data storage unit, the inertial sensing unit, the ambient light detection circuit, the wristband detection circuit, the impedance detection circuit and the electrical stimulation circuit are all electrically connected with the microprocessor; the circuit board is also provided with a high-voltage circuit, and the high-voltage circuit is connected with the circuit management unit and the electrical stimulation circuit; and the communication unit is connected with the circuit management unit.

Preferably, the bottom of the shell is provided with a limiting slot matched with the base; the adapter port and the plurality of magnetic attraction slots are located in the limiting slot; a bottom edge of the shell is a rounded corner; an inclined surface gradually rising from inside to outside is reserved between the rounded corner and the limiting slot; and corresponding spring bar seats are arranged on front and rear end surfaces of the shell.

Preferably, the outer band includes a fabric band and a first connecting lug; two ends of the fabric band are respectively connected with a second connecting lug and a tail knot; the first connecting lug is provided with a rectangular hole: one end of the fabric band close to the tail knot is adhered to an outer side of the fabric band through a hook and loop fastener after passing through the rectangular hole, wherein the first connecting lug and the second connecting lug are respectively movably connected with the spring bar seats on the front and rear sides of the fixed main unit.

Preferably, a reinforcing cover plate is covered on the base; a side end of the base is connected with a charging interface cover; a round iron sheet slot is arranged on the charging port cover; a round iron sheet is embedded in the round iron sheet slot; an upper surface of the round iron sheet overlaps a surface of the charging interface cover; the upper surface of the round iron sheet is connected to the magnets embedded in the charging interface in an attracting manner; the adapter seat is provided with a plurality of pogopins; and the pogopins on the adapter seat are in contact connection with the pogopins on the adapter port.

A use method of the wristband nerve stimulator includes:
S1: mounting the fixed main unit and the base in a cooperating manner; plugging the base in the limiting slot; embedding the adapter seat in the adapter port; connecting the pogopins on the adapter seat to the pogopins on the adapter port in a contact manner; electrically connecting the fixed main unit to the electrode assembly; connecting the magnets in the magnetic attraction slots with the magnets in the round magnetic attraction slots in a mutual attracting manner; mounting and fixing the fixed main unit with the base on the inner band: wrapping the inner side of the outer band by the inner band; fastening the fasteners on the outer band;
S2: wearing the outer band and the inner band on the wrist; tensioning one end of the fabric band close to the tail knot to enable the ring formed by the fixed main unit and the outer band to retract towards the inner side in a diameter direction, so that the electrode pieces on the electrode assembly are connected to the skin of a user in a contact manner; fitting the outer band to the inner band; adhering the end of the fabric band close to the tail knot to the outer side of the fabric band through the hook and loop fastener; and
S3: pressing the buttons at the side end of the shell to start and select functions, outputting power of the lithium battery through the circuit board to the electrode assembly through the adapter port and the adapter seat, and outputting a personalized stimulation mode and pulse parameters for treatment by the electrode pieces according to physical signs of different users.

Compared with the prior art, the present disclosure has the following beneficial effects.

By the arrangement of the fixed main unit and the base, the base is plugged into the limiting slot, and the adapter seat is embedded in the adapter port; the pogopins on the adapter seat are connected to the pogopins on the adapter port in a contact manner; the magnets in the magnetic attraction slots are connected with the magnets in the round magnetic attraction slots in a mutual attracting manner; and the fixed main unit and the band are mounted and fixed, and the fasteners are fastened on the outer band, so that the inner band, the fixed main unit and the outer band can be quickly mounted.

By the arrangement of the shell, the shell and the screen assembly, the shell and the buttons, the shell and the charging interface, and the shell and the adapter port are all sealed by the sealants, so that the fixed main unit can have a waterproof effect, and the overall waterproofness of the nerve stimulator is improved.

By the arrangement of the band, an inner band with electrode pieces having a matching length and matching curvature can be selected according to a diameter and curvature of the wrist of a user, and the electrode pieces on the inner band can be fully fitted to the skin of the user and fixed by means of adjusting the outer band.

The charging interface of the present disclosure is arranged at the bottom of the shell. Since the wrist shields the charging interface, the user cannot use this product while charging this product, and the safety of the product is improved.

### BRIEF DESCRIPTION OF THE DRAWINGS

FIG. 1 is a schematic structural diagram of the present disclosure;

FIG. 2 is a schematic structural diagram of a fixed main unit of the present disclosure;

FIG. 3 is a schematic exploded diagram of a fixed main unit of the present disclosure;

FIG. 4 is a partially sectional view of a shell of the present disclosure;

FIG. 5 is a schematic structural diagram of a screen assembly of the present disclosure;

FIG. 6 is a schematic structural diagram of a band of the present disclosure;

FIG. 7 is a schematic exploded diagram of a band of the present disclosure;

FIG. 8 is a schematic structural diagram of an outer band of the present disclosure;

FIG. 9 is a diagram I of a use state of the present disclosure;

FIG. 10 is a diagram II of a use state of the present disclosure;

FIG. 11 is a diagram III of a use state of the present disclosure;

FIG. 12 is a diagram IV of a use state of the present disclosure;

FIG. 13 is a working principle block diagram of the present disclosure;

FIG. 14 is a working principle flow diagram of the present disclosure;

FIG. 15 is a working principle diagram of an electrical stimulation circuit of the present disclosure; and

FIG. 16 is a working flow diagram of an electrical stimulation circuit of the present disclosure.

### DETAILED DESCRIPTION OF THE EMBODIMENTS

The technical solutions in the embodiments of the present disclosure will be described clearly and completely below in combination with the accompanying drawings of the embodiments of the present disclosure. Apparently, the described embodiments are only part of the embodiments of the present disclosure, not all embodiments. All other embodiments obtained by those of ordinary skill in the art based on the embodiments in the present disclosure without creative work shall fall within the protection scope of the present disclosure.

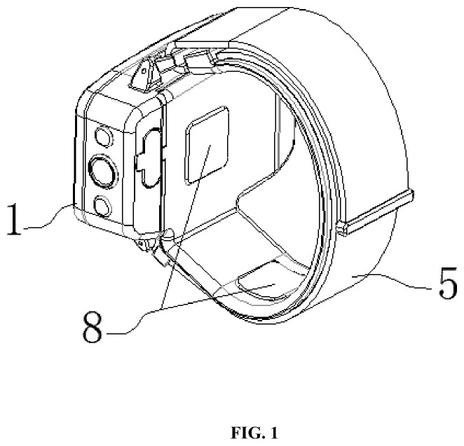

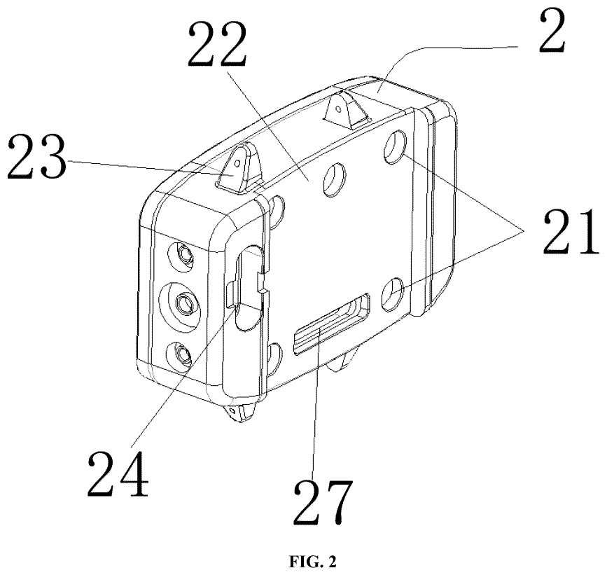

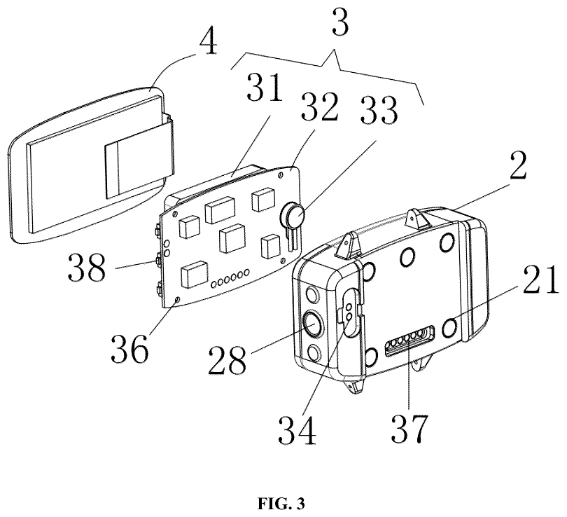

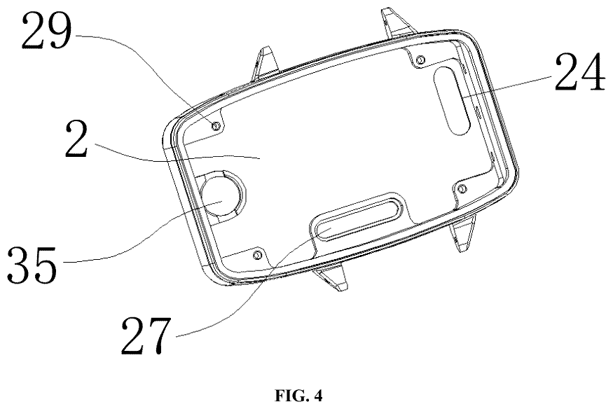

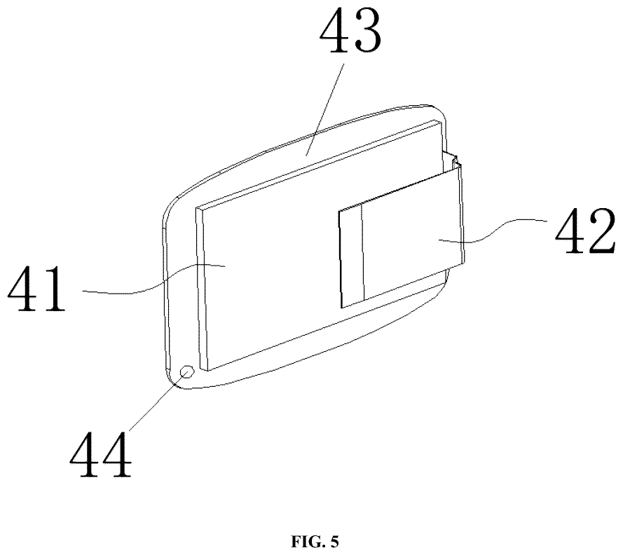

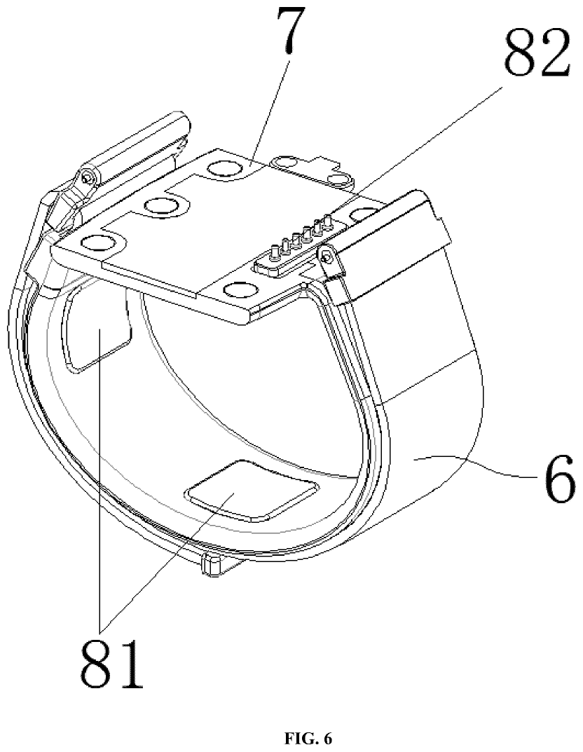

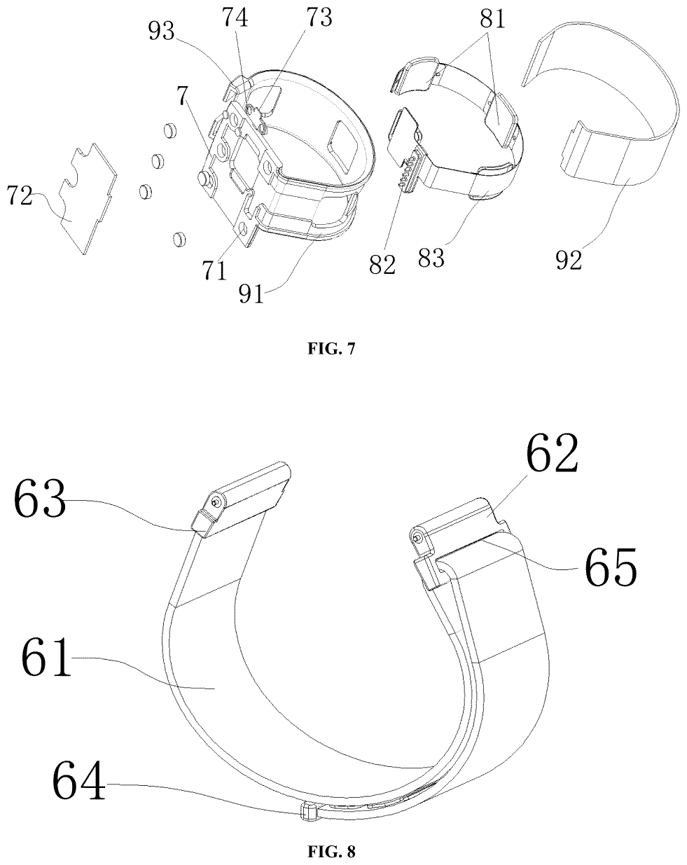

Referring to FIG. 1 to FIG. 8, an embodiment provided by the present disclosure is shown: A wristband nerve stimulator includes:
a fixed main unit 1, wherein the fixed main unit 1 includes a shell 2, a circuit assembly 3 and a screen assembly 4; the circuit assembly 3 is electrically connected with the screen assembly 4; the screen assembly 4 and the shell 2 are encapsulated through a waterproof sealing rubber strip; the circuit assembly 3 is fixedly mounted in the shell 2; the circuit assembly 3 includes a lithium battery 31, a circuit board 32, and a vibration motor 33 arranged on the circuit board 32; a plurality of first threaded holes 29 and a framework 35 matched with the vibration motor 33 are arranged in the shell 2; the vibration motor 33 is embedded into the framework 35; a plurality of second threaded holes 36 corresponding to the first threaded holes 29 are formed in four corners of the circuit board 32; the plurality of first threaded holes 29 are fixedly connected with the plurality of second threaded holes 36 through screws; the first threaded holes 29 are all blind holes; a charging interface 34 and an adapter port 37 are arranged on the circuit board 32; a bottom of the shell 2 is provided with a charging interface location 24 and an adapter port location 27 which are matched with the charging interface 34 and the adapter port 37; the charging interface 34 and the adapter port 37 are respectively embedded into the charging interface location 24 and the adapter port location 27; spaces between the charging interface 34 and the charging interface location 24 as well as between the adapter port 37 and the adapter port location 27 are encapsulated by sealants; the bottom of the shell 2 is also provided with a plurality of magnetic attraction slots 21; a magnet is embedded into each magnetic attraction slot 21, wherein the fixed main unit 1 is wirelessly connected with electronic equipment through Bluetooth;
a band 5, wherein the band 5 includes an outer band 6 and an inner band 10; the inner band 10 includes a base 7 and an electrode assembly 8; the base 7 is connected with the electrode assembly 8; the electrode assembly 8 extends to the outside from one side of the base; the electrode assembly 8 includes a plurality of electrode pieces 81 and an adapter seat 82; the plurality of electrode pieces 81 are sequentially connected through an FPC flat cable 83; the electrode piece 81 at the outermost end is arranged at the bottom of the base 7 and protrudes from the bottom of the base 7, and the electrode pieces 81 are planar electrode pieces: sealing layers 9 wrap outer sides of the remaining electrode pieces 81 and the FPC flat cable 83; the electrode pieces 84 on the sealing layers 9 protrude from inner walls of sealing strips 9; an open fastener 93 is arranged on one side of each sealing layer 9 away from the base 7; the fastener 93 is composed of two opposite “ [symbol] ” shapes; the fastener 93 is fastened on the outer band 6; the fastener 93 is made of a soft rubber material; the adapter seat 82 is arranged on the base 7; the adapter seat 82 is electrically connected with the FPC flat cable 83; the base 7 is also provided with a plurality of circular magnetic attraction slots 71 matched with the plurality of magnetic attraction slots 21; a magnet is embedded in each circular magnetic attraction slot 71; the magnets in the plurality of magnetic attraction slots 21 are connected to the magnets in the plurality of circular magnetic attraction slots 71 in an attracting manner; the adapter seat 82 is embedded into the adapter port 37; and the circuit assembly 3 is electrically connected with the electrode assembly 8.

Each sealing layer 9 includes an inner band main body 91 and an outer adhesive layer 92. The FPC flat cable 83 is arranged between the inner band main body 91 and the outer adhesive layer 92. The inner band main body 91 and the outer adhesive layer 92 are hermetically connected and is wrapped by the FPC flat cable 83. The electrode pieces 84 penetrate through the outer adhesive layers 92.

Preferably; the screen assembly 4 includes a display screen 41 and a display interface flat cable 42; the display screen 41 is connected with the circuit board 32 through the display interface flat cable 42; a glass cover plate 43 is covered on the display screen 41; and a transparent frosted region 44 is arranged at one corner of the glass cover plate 43.

Preferably, a plurality of micro switches 38 are arranged at a side end of the circuit board 32; a plurality of buttons 28 matched with the micro switches 38 are arranged at a side end of the shell 2; the buttons 28 and the shell 2 linearly slide and are in clearance fit; a reset spring (not shown) and a waterproof sealing ring (not shown) are arranged between the buttons 28 and the shell 2; the plurality of micro switches 38 resist against the plurality of buttons 28; a charging abutment contact 341 and an adapter port abutment contact 371 are also arranged on the circuit board 32; the charging abutment contact 341 is connected with the charging interface 34; a plurality of pogopins are arranged on the adapter port 37; the pogopins on the adapter port 37 are connected with the adapter port abutment contact 371 in a contacting manner; and the shell 2 and the screen assembly 4, the shell 2 and the buttons 28, the shell 2 and the charging interface 34, and the shell 2 and the adapter port 37 are all sealed by the sealants, so that the fixed main unit 1 can have a waterproof effect, and the overall waterproofness of the nerve stimulator is improved.

Preferably, the bottom of the shell 2 is provided with a limiting slot 22 matched with the base 7; the adapter port 37 and the plurality of magnetic attraction slots 21 are located in the limiting slot 22; a bottom edge of the shell 2 is a rounded corner; an inclined surface gradually rising from inside to outside is reserved between the rounded corner and the limiting slot; and corresponding spring bar seats 23 are arranged on front and rear end surfaces of the shell 2.

Preferably, the outer band 6 includes a fabric band 61 and a first connecting lug 62; two ends of the fabric band 61 are respectively connected with a second connecting lug 63 and a tail knot 64; the first connecting lug 62 is provided with a rectangular hole 65; one end of the fabric band 61 close to the tail knot 64 is adhered to an outer side of the fabric band 61 through a hook and loop fastener after passing through the rectangular hole 65, wherein the first connecting lug 62 and the second connecting lug 63 are respectively movably connected with the spring bar seats 23 on the front and rear sides of the fixed main unit 1 through rotating shafts.

Preferably, a reinforcing cover plate 72 is covered on the base 7; a side end of the base 7 is connected with a charging interface cover 73; a round iron sheet slot 74 is arranged on the charging port cover 73; a round iron sheet is embedded in the round iron sheet slot 74; an upper surface of the round iron sheet overlaps a surface of the charging interface cover; the upper surface of the round iron sheet is connected to the magnets embedded in the charging interface in an attracting manner; the adapter seat 82 is provided with a plurality of pogopins; and the pogopins on the adapter seat 82 are in contact connection with the pogopins on the adapter port 37.

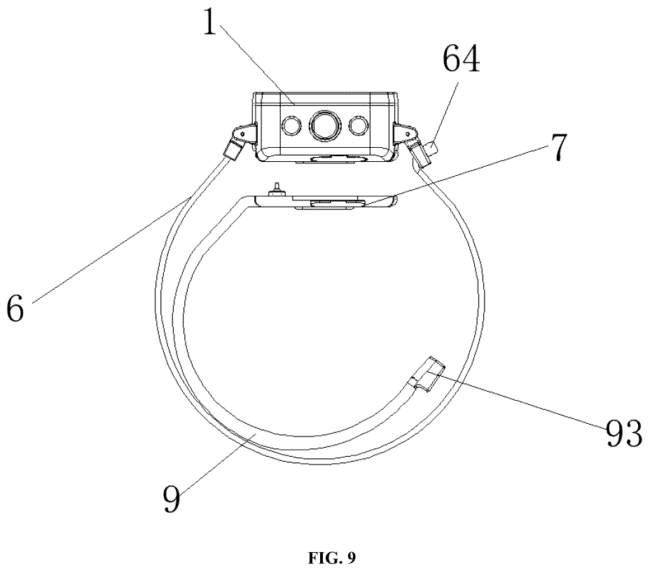

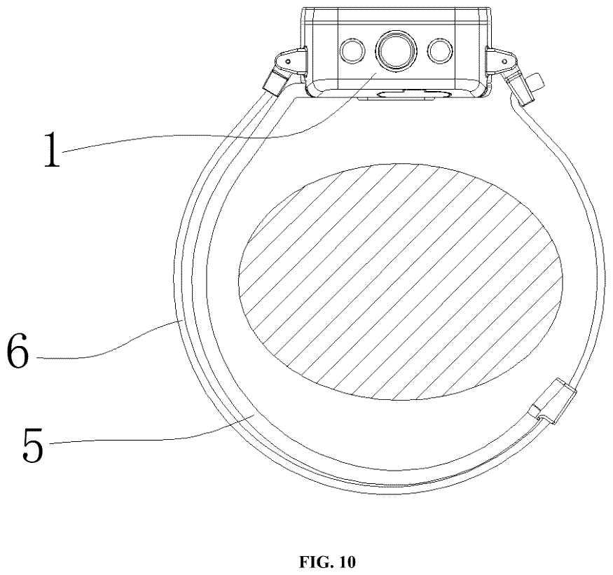

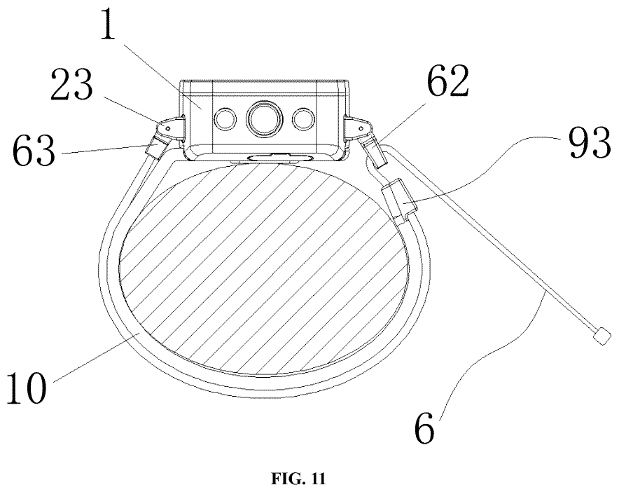

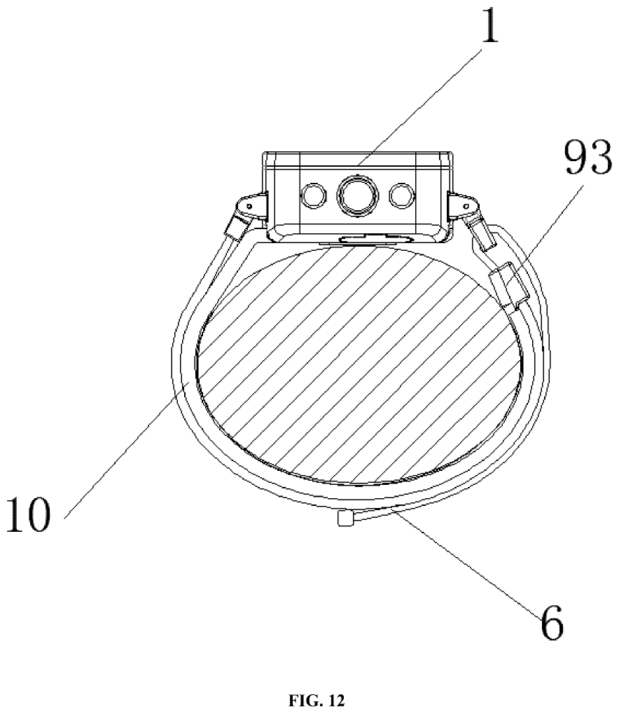

Referring to FIG. 9 to FIG. 12; a use method of the wristband nerve stimulator includes:
S1; mounting the fixed main unit 1 and the base 7 in a cooperating manner; plugging the base 7 in the limiting slot 22; embedding the adapter seat 82 in the adapter port 37; connecting the pogopins on the adapter seat 82 to the pogopins on the adapter port 37 in a contact manner; electrically connecting the fixed main unit 1 to the electrode assembly 8; connecting the magnets in the magnetic attraction slots 21 with the magnets in the round magnetic attraction slots 12 in a mutual attracting manner; mounting and fixing the fixed main unit 1 with the base 7 on the inner band 10; wrapping the inner side of the outer band 6 by the inner band 10; fastening the fasteners 93 on the outer band 6;
S2: wearing the outer band 6 and the inner band 10 on the wrist; tensioning one end of the fabric band 63 close to the tail knot 64 to enable the ring formed by the fixed main unit 1 and the outer band 6 to retract towards the inner side in a diameter direction, so that the electrode pieces 81 on the electrode assembly 8 are connected to the skin of a user in a contact manner; fitting the outer band 6 to the inner band 10; adhering the end of the fabric band 61 close to the tail knot 64 to the outer side of the fabric band 61 through the hook and loop fastener; and
S3; pressing the buttons 28 at the side end of the shell 2 to start and select functions, outputting power of the lithium battery 31 through the circuit board 32 to the electrode assembly 8 through the adapter port 37 and the adapter seat 11, and outputting a personalized stimulation mode and pulse parameters for treatment by the electrode pieces 81 according to physical signs of different users.

The magnets in the magnetic attraction slots 21 are connected to the magnets in the circular magnetic attraction slots 12 in the mutual attracting manner, and the base 7 and the limiting slot 22 are mounted cooperatively, so that the inner band 10 and the fixed main unit 1 are quickly mounted. For removal, the inner band 10 can be directly pulled away. This is convenient for the replacement of the inner band 10.

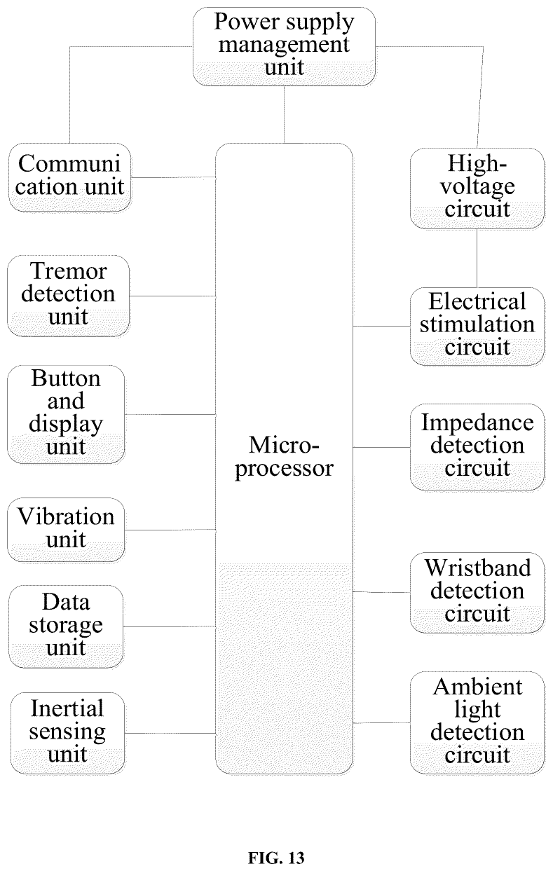

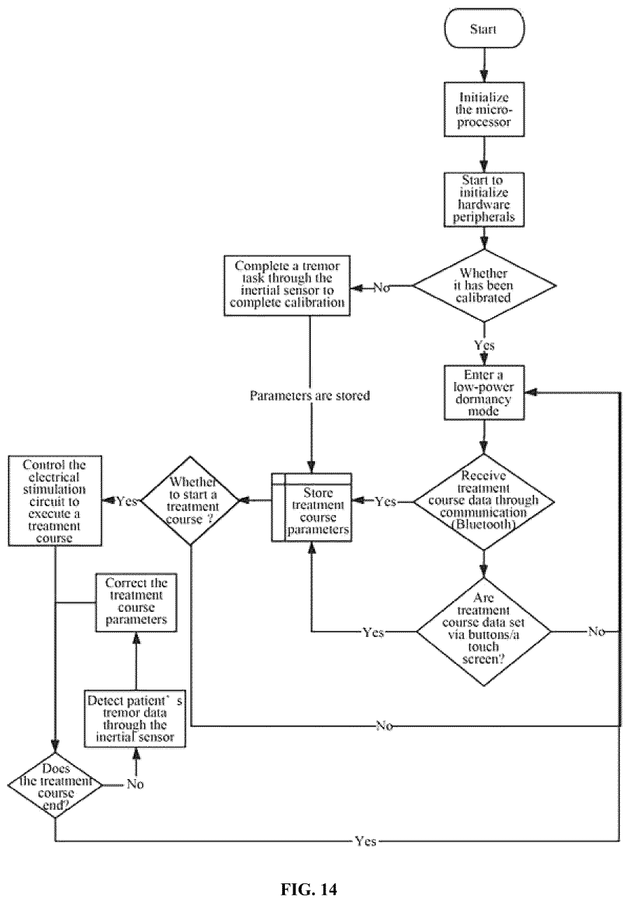

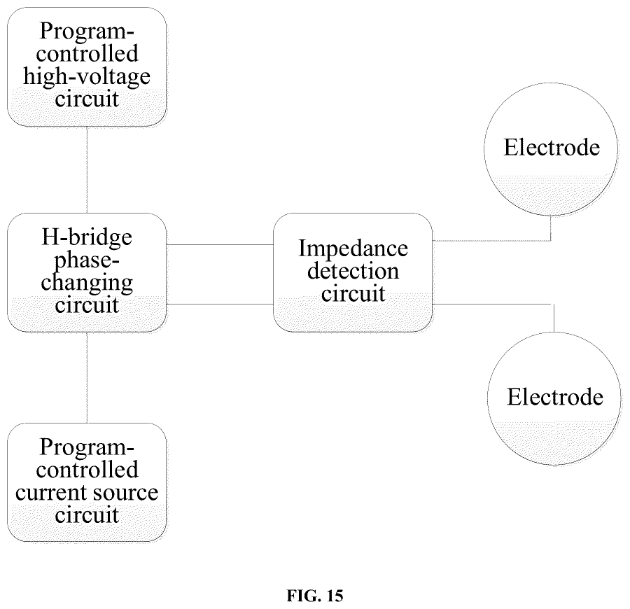

Referring to FIG. 13 to FIG. 14, the circuit board 32 includes a microprocessor, an circuit management unit, a communication unit, a tremor detection unit, a button and display unit, a vibration unit, a data storage unit, an inertial sensing unit, an ambient light detection circuit, a wristband detection circuit, an impedance detection circuit and an electrical stimulation circuit; the circuit management unit, the communication unit, the tremor detection unit, the button and display unit, the vibration unit, the data storage unit, the inertial sensing unit, the ambient light detection circuit, the wristband detection circuit, the impedance detection circuit and the electrical stimulation circuit are all electrically connected with the microprocessor; the circuit board is also provided with a high-voltage circuit, and the high-voltage circuit is connected with the circuit management unit and the electrical stimulation circuit; and the communication unit is connected with the circuit management unit.

There are two working flows of the wristband nerve stimulator. The first working flow has been calibrated, and the second working flow is not calibrated. Details are described below:

After the wristband nerve stimulator is started, the microprocessor is initialized, and hardware peripherals of the fixed main unit 1 start to be initialized. The screen assembly 4 displays whether it has been calibrated. If the button 28 is pressed to select “Yes”, the fixed main unit 1 enters a low-power dormancy mode. The fixed main unit 1 will store the treatment course data when it receives treatment course data through Bluetooth. When the fixed main unit 1 has no treatment course data, treatment course data can be set by the buttons or a touch screen. When the screen assembly 4 displays whether to start a treatment course? If the button 28 is pressed to select “Yes”, the microprocessor controls the electrical stimulation circuit to carry out the treatment course; and if the button 28 is pressed to select “No”, the fixed main unit 1 enters the low-power dormancy mode. When the electrical stimulation course ends, the fixed main unit 1 enters the low-power dormancy mode. When the electrical stimulation course does not end, patient's tremor data will be monitored in real time through the inertial sensor, and parameters of the treatment course will be corrected in real time until the treatment course ends.

After the wristband nerve stimulator is started, the microprocessor is initialized, and the hardware peripherals of the fixed main unit 1 start to be initialized. The screen assembly 4 displays whether it has been calibrated. The button 28 is pressed to select “No”. A tremor task is completed through the tremor detection unit, and the treatment course parameters are stored. The screen assembly 4 displays whether to start a treatment course. If the button 28 is pressed to select “Yes”, the microprocessor controls the electrical stimulation circuit to carry out the treatment course; and if the button 28 is pressed to select “No”, the fixed main unit 1 enters the low-power dormancy mode. When the electrical stimulation course ends, the fixed main unit 1 enters the low-power dormancy mode. When the electrical stimulation course does not end, patient's tremor data will be monitored in real time through the inertial sensor, and parameters of the treatment course will be corrected in real time until the treatment course ends.

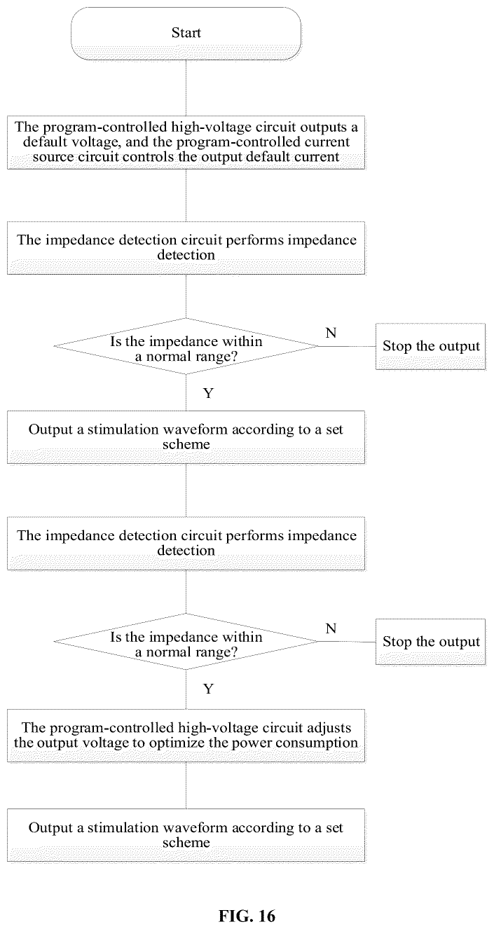

Referring to FIG. 15 to FIG. 16, the program-controlled high-voltage circuit is connected to a high end of an H-bridge phase-changing circuit as a stimulation power supply. A low end of the H-bridge phase-changing circuit is connected to a voltage-controlled constant current source. The voltage-controlled constant current source controls a stimulation current. The impedance detection circuit is connected in series between the H-bridge phase-changing circuit and electrodes for load impedance detection.

The working flow of the stimulation circuit is as follows: At the beginning, the program-controlled high-voltage circuit outputs a default voltage, and the program-controlled current source circuit controls the output default current; the impedance detection circuit performs impedance detection to detect whether the impedance is within a normal range; and if the measured impedance is within the normal range, a stimulation waveform is output according to a set scheme, otherwise, the output is stopped. During the stimulation, the impedance detection circuit performs real-time impedance detection, and adjusts an output of the high-voltage source according to detection results to optimize the power consumption. When the impedance is beyond the normal range, the stimulation is stopped.

For those skilled in the art, it is apparent that the present disclosure is not limited to the details of the demonstrative embodiments mentioned above, and that the present disclosure can be realized in other specific forms without departing from the spirit or basic features of the present disclosure. Therefore, from any point of view, the embodiments should be regarded as exemplary and non-limiting. The scope of the present disclosure is defined by the appended claims rather than the above description. Therefore, all changes falling within the meanings and scope of equivalent elements of the claims are intended to be included in the present disclosure. No drawing markings in claims shall be deemed to limit the claims involved.

What is claimed is:

1. A wristband nerve stimulator, comprising:
a fixed main unit, wherein the fixed main unit comprises a shell, a circuit assembly and a screen assembly; the circuit assembly is electrically connected with the screen assembly; the screen assembly and the shell are encapsulated through a waterproof sealing rubber strip; the circuit assembly is fixedly mounted in the shell; the circuit assembly comprises a lithium battery, a circuit board, and a vibration motor arranged on the circuit board; a plurality of first threaded holes and a framework matched with the vibration motor are arranged in the shell; the vibration motor is embedded into the framework; a plurality of second threaded holes corresponding to the first threaded holes are formed in four corners of the circuit board; the plurality of first threaded holes are fixedly connected with the plurality of second threaded holes through screws; the first threaded holes are all blind holes; a charging interface and an adapter port are arranged on the circuit board; a bottom of the shell is provided with a charging interface location and an adapter port location which are matched with the charging interface and the adapter port; the charging interface and the adapter port are respectively embedded into the charging interface location and the adapter port location; spaces between the charging interface and the charging interface location as well as between the adapter port and the adapter port location are encapsulated by sealants; the bottom of the shell is also provided with a plurality of magnetic attraction slots; a magnet is embedded into each magnetic attraction slot;
a band, wherein the band comprises an outer band and an inner band; the inner band comprises a base and an electrode assembly; the base is connected with the electrode assembly; the electrode assembly comprises a plurality of electrode pieces and an adapter seat; the plurality of electrode pieces are sequentially connected through a flexible printed circuit (FPC) flat cable; the electrode piece at the outermost end is arranged at the bottom of the base and protrudes from the bottom of the base, and sealing layers wrap outer sides of the remaining electrode pieces and the FPC flat cable; the electrode pieces on the sealing layers protrude from inner walls of sealing strips; an open fastener is arranged on one side of each sealing layer away from the base; the fastener is fastened on the outer band; the adapter seat is arranged on the base; the adapter seat is electrically connected with the FPC flat cable; the base is also provided with a plurality of circular magnetic attraction slots matched with the plurality of magnetic attraction slots; a magnet is embedded in each circular magnetic attraction slot; the magnets in the plurality of magnetic attraction slots are connected to the magnets in the plurality of circular magnetic attraction slots in an attracting manner; the adapter seat is embedded into the adapter port; the circuit assembly is electrically connected with the electrode assembly; two ends of the outer band are movably connected with front and rear sides of the fixed main unit; the outer band and the fixed main unit form a ring; and the ring wraps the inner band.

2. The wristband nerve stimulator according to claim 1, wherein the screen assembly comprises a display screen and a display interface flat cable; the display screen is connected with the circuit board through the display interface flat cable; a glass cover plate is covered on the display screen; and a transparent frosted region is arranged at one corner of the glass cover plate.

3. The wristband nerve stimulator according to claim 1, wherein a plurality of micro switches are arranged at a side end of the circuit board; a plurality of buttons matched with the micro switches are arranged at a side end of the shell; the buttons and the shell linearly slide and are in clearance fit; a reset spring and a waterproof sealing ring are arranged between the buttons and the shell; the plurality of micro switches resist against the plurality of buttons; a charging abutment contact and an adapter port abutment contact are also arranged on the circuit board; the charging abutment contact is connected with the charging interface; a plurality of pogopins are arranged on the adapter port; the pogopins on the adapter port are connected with the adapter port abutment contact in a contacting manner; and two magnets are embedded in the charging interface.

4. The wristband nerve stimulator according to claim 1, wherein the circuit board comprises a microprocessor, a circuit management unit, a communication unit, a tremor detection unit, a button and display unit, a vibration unit, a data storage unit, an inertial sensing unit, an ambient light detection circuit, a wristband detection circuit, an impedance detection circuit and an electrical stimulation circuit; the circuit management unit, the communication unit, the tremor detection unit, the button and display unit, the vibration unit, the data storage unit, the inertial sensing unit, the ambient light detection circuit, the wristband detection circuit, the impedance detection circuit and the electrical stimulation circuit are all electrically connected with the microprocessor; the circuit board is also provided with a high-voltage circuit, and the high-voltage circuit is connected with the circuit management unit and the electrical stimulation circuit; and the communication unit is connected with the circuit management unit.

5. The wristband nerve stimulator according to claim 1, wherein the bottom of the shell is provided with a limiting slot matched with the base; the adapter port and the plurality of magnetic attraction slots are located in the limiting slot; a bottom edge of the shell is a rounded corner; an inclined surface gradually rising from inside to outside is reserved between the rounded corner and the limiting slot; and corresponding spring bar seats are arranged on front and rear end surfaces of the shell.

6. The wristband nerve stimulator according to claim 1, wherein the outer band comprises a fabric band and a first connecting lug; two ends of the fabric band are respectively connected with a second connecting lug and a tail knot; the first connecting lug is provided with a rectangular hole; one end of the fabric band close to the tail knot is adhered to an outer side of the fabric band through a hook and loop fastener after passing through the rectangular hole, wherein the first connecting lug and the second connecting lug are respectively movably connected with the spring bar seats on the front and rear sides of the fixed main unit.

7. The wristband nerve stimulator according to claim 1, wherein a reinforcing cover plate is covered on the base; a side end of the base is connected with a charging interface cover; a round iron sheet slot is arranged on the charging port cover; a round iron sheet is embedded in the round iron sheet slot; an upper surface of the round iron sheet overlaps a surface of the charging interface cover; the upper surface of the round iron sheet is connected to the magnets embedded in the charging interface in an attracting manner; the adapter seat is provided with a plurality of pogopins; and the pogopins on the adapter seat are in contact connection with the pogopins on the adapter port.

8. A use method of the wristband nerve stimulator, comprising:
S1: mounting the fixed main unit and the base in a cooperating manner; plugging the base in the limiting slot; embedding the adapter seat in the adapter port; connecting the pogopins on the adapter seat to the pogopins on the adapter port in a contact manner; electrically connecting the fixed main unit to the electrode assembly; connecting the magnets in the magnetic attraction slots with the magnets in the round magnetic attraction slots in a mutual attracting manner; mounting and fixing the fixed main unit with the base on the inner band; wrapping the inner side of the outer band by the inner band; fastening the fasteners on the outer band;
S2: wearing the outer band and the inner band on the wrist; tensioning one end of the fabric band close to the tail knot to enable the ring formed by the fixed main unit and the outer band to retract towards the inner side in a diameter direction, so that the electrode pieces on the electrode assembly are connected to the skin of a user in a contact manner; fitting the outer band to the inner band; adhering the end of the fabric band close to the tail knot to the outer side of the fabric band through the hook and loop fastener; and
S3: pressing the buttons at the side end of the shell to start and select functions, outputting power of the lithium battery through the circuit board to the electrode assembly through the adapter port and the adapter seat, and outputting a personalized stimulation mode and pulse parameters for treatment by the electrode pieces according to physical signs of different users.
